(19) United States
(12) Patent Application Publication
(10) Pub. No.: US 2024/0284877 A1
(43) Pub. Date: Aug. 29, 2024

(54) IMPROVED METHOD FOR DETERMINING THE SEX OF A CHICK

(71) Applicant: EGG-CHICK AUTOMATED TECHNOLOGIES, LANDIVISIAU (FR)

(72) Inventors: Hamdi BOUKAMCHA, LANDIVISIAU (FR); Thierry CHAPELET, LANDIVISIAU (FR)

(21) Appl. No.: 18/573,496

(22) PCT Filed: Jul. 4, 2022
(86) PCT No.: PCT/FR2022/051328

§ 371 Date: Dec. 22, 2023

(30) Foreign Application Priority Data
Jul. 8, 2021 (FR) FR2107408

(51) Int. Cl. A01K 45/00; G06V 10/25; G06V 10/44; G06V 10/48; G06V 10/74; G06V 10/764; G06V 10/774; G06V 40/10

(52) U.S. Cl. CPC A01K 45/00; G06V 10/25; G06V 10/446; G06V 10/48; G06V 10/761; G06V 10/764; G06V 10/774; G06V 40/10

(57) ABSTRACT

The invention relates to a method for determining the sex of a chick, comprising: determining (100) a region of interest in the image in which the feathers of a wing are visible, and running, on said region of interest, a classification model (400) trained on a training data set comprising images of male chick wings and female chick wings, in order to determine whether the chick is male or female.

### TECHNICAL FIELD

The present invention relates to a method for determining the sex of the chicks by observing the arrangement of the feathers at the wing tips, and a device for implementing this method.

### PRIOR ART

It is known to determine the sex of the chicks as a function of the arrangement of the feathers, located at the ting tips. Indeed, as can be seen in FIGS. 1a and 1b, the wing tips comprise two rows of feathers which are the primaries and the coverts. In the Figures, the tips of the primaries and coverts, respectively, were highlighted. In male chicks, in FIG. 1a, these two rows of feathers terminate at the same level, while in female chicks, in FIG. 1b, the tips of the primaries terminate further than the tips of the coverts, and it is therefore possible to observe an alternation of relatively short and long feathers.

Determining the sex of the chicks by the feathers was initially carried out by manually an operator, which requires significant time.

Solutions have also been proposed to automate this determination.

Thus, document EP 1-092-347 discloses a method for determining the sex of the chicks which uses a conveyor making it possible to separate the chicks and to bring them near a camera. At this point, the check is unbalanced, for example by vibrations of the conveyor, to cause it to deploy its wings at its own initiative, and one or more images of the chick's wings are acquired.

Image processing methods have also been proposed to automatically determine the sex of the chick from the acquired image. For example, in document U.S. Pat. No. 6,396,938, a first method comprises the extraction of shape parameters from each feather (length, position of the medium, and position of the tip), and the calculation of a parameter that quantifies the length variation of two adjacent feathers.

Another method described in this document comprises the location of the coordinates of the tips of the feathers, and the determination of a polynomial function connecting the tips of the feathers. The sex of the chick is determined as a function of the parameters of the polynomial function.

The chicks for which the result is uncertain can be studied manually or subject to a new automatic determination.

However, in document U.S. Pat. No. 6,396,938, these methods are implemented on a previously annotated chick wing image to identify at least the tips of the feathers. The methods described do not therefore make it possible to implement a fully automatic processing of determining the sex of the chick from the capture of the image.

### SUMMARY

Considering the foregoing, the aim of the invention is to propose an improved, entirely automatic solution for determining the sex of a chick from an image of a wing of the chick.

In particular, the aim of the invention is to propose fast, reliable processing of an image in order to determine the sex of the chick.

Another purpose of the invention is to propose fully automated processing for determining the sex of the chick.

In this respect, the invention relates to a method for determining the sex of a chick, the method being implemented by computer from an image of a chick, the method comprising:
determining a region of interest of the image on which the feathers of a wing are visible,
running, on said region of interest, a classification model trained on a training data set comprising images of male chick wings and of female chick wings, to determine the male or female sex of the chick.

In some embodiments, the method is implemented for each of a plurality of images acquired on a same chick, and further comprises a step of determining the sex of the chick from the results obtained by the classification model for all of the images.

In some embodiments, determining a region of interest of the image comprises:
scanning the image with a window of determined size to define a plurality of regions of the image,
for each region, calculating a Haar feature of the region,
the application on each Haar feature of a trained classifier to determine whether or not the region represents feathers, and
determining a region of interest of the image as a region representing feathers.

In some embodiments, the method further comprises processing the region of interest to determine a set of lines corresponding to the feathers of the chick on the image, determining a set of parameters from the extracted lines, and the classification model is applied to said set of parameters.

In some embodiments, the processing of the region of interest to determine a set of lines corresponding to the feathers on the image comprises:
running an edge detection processing on the region of interest, and
applying, to the edges resulting from the processing, a Hough transform to determine a set of lines corresponding to the feathers visible on the region of interest.

In some embodiments, the edge detection processing comprises:
running a Gaussian filter and thresholding the region of interest in order to obtain a binary representation of the region of interest,
calculating a distance map on the binary representation of the region of interest, to determine a distance between each point and an edge closest to said point, and normalizing said map to obtain a grayscale representation of the region of interest, and
running an erosion operation on the obtained grayscale representation.

In some embodiments, thresholding the region of interest comprises the determination, for each current pixel of the region of interest, of a thresholding value determined as a function of the intensity values of the pixels included in a local vicinity of the current pixel.

In some embodiments, determining all of the lines representing the feathers further comprises:
rotating the region of interest by an angle determined from the angle, relative to the horizontal, of the longest lines, to make said lines substantially horizontal, and
eliminating the lines which, after rotating the region of interest, extend in a direction forming an angle greater than a predetermined threshold relative to the x-axis.

In some embodiments, determining the parameters from the extracted lines comprises:
identifying a set of lines corresponding to long feathers, and
identifying a set of lines corresponding to short feathers.

In some embodiments, the method comprises implementing a rotation of the region of interest so that the lines representing the feathers extend substantially horizontally, ranking each line in order of length, and identifying the set of lines corresponding to long feathers comprises:
initializing the set of lines corresponding to long feathers, said set comprising the longest line,
implementing, for each line included in said set, the following steps:
   identifying all the neighboring lines of the considered line along the vertical axis,
   calculating, for each neighboring line, a length difference and a distance between the center of the neighboring line and the center of the line in question,
   if the relative difference and the distance are less than respective thresholds, identifying the neighboring line as a line corresponding to a long feather, and adding to the set of lines corresponding to long feathers.

In some embodiments, identifying a set of lines corresponding to short feathers comprises implementing, for each line corresponding to a long feather of the set, starting with the line located at the maximum vertical position of the set, the following steps:
identifying, among the lines not belonging to the set of lines corresponding to long feathers, the neighboring lines of the considered line,
calculating, for each neighboring line, a length difference, a distance along the vertical axis between the considered line and the neighboring line, and a distance along the horizontal axis between a distal end, respectively proximal end, of the considered line, and the proximal end, respectively distal end, of the neighboring line,
if the calculated differences and distances are less than respective thresholds, identifying the neighboring line as a line corresponding to a short feather.

In some embodiments, the parameters determined from the lines comprise at least:
a number of lines corresponding to long feathers,
a number of lines corresponding to short feathers,
an average angle between the lines and the horizontal, and
an average deviation, measured vertically, between two adjacent lines.

In some embodiments, the parameters determined from the lines further comprise at least one of the group consisting of:
the minimum, maximum, and/or average horizontal and/or vertical positions of the centers of the lines,
the minimum, average and/or maximum horizontal and/or vertical distance between the centers of two consecutive lines,
the minimum, average and/or maximum lengths of the lines corresponding to long feathers,
the minimum, average and/or maximum lengths of the lines corresponding to short feathers,
the average intensity of the pixels of a line corresponding to a short feather,
the average intensity of the pixels of a line corresponding to a long feather,
the difference in average intensity of the pixels between the lines corresponding to long feathers and the lines corresponding to short feathers,
the location relative to the horizontal axis of each line, a minimum, average and/or maximum angle between two lines,
a minimum, average and/or maximum distance between the proximal ends of two lines corresponding to a long feather and one line corresponding to a short feather located between them,
a minimum, average and/or maximum distance between the distal ends of two lines corresponding to a long feather and one line corresponding to a short feather located between them.

In some embodiments, the classification model trained to determine the sex of a chick is a decision tree.

In some embodiments, the classification model is trained on a database of annotated training images, where each training image is obtained by applying steps of determining a region of interest and processing the region of interest to extract a set of lines representing the feathers, and the annotation comprises an indication of the sex of the chick and an associated certainty level, determined from a number of lines corresponding to long feathers and a number of lines corresponding to short feathers.

In some embodiments, the method is implemented on a set of images of the same chick, and comprises determining the sex of the chick from the result most frequently provided by the classification model.

According to another subject matter, a computer program product is described, comprising code instructions for implementing the method according to the preceding description, when it is executed by a computing unit.

The invention also relates to a device for determining the sex of a chick comprising at least:
a camera adapted to acquire at least one image of a chick, and
a computing unit configured to implement the method according to the preceding description on the image acquired by the camera.

In some embodiments, the camera is adapted to acquire images in a wavelength range between 340 and 500 nm, preferably between 400 and 450 nm.

In some embodiments, the device further comprises a conveyor adapted to bring a chick into the field of view of the camera, wherein the conveyor is adapted to unbalance the chicks so that the chick has its wings unfurled when it is in front of the camera.

In some embodiments, the camera is configured to acquire a series of at least 20 images of each chick.

In some embodiments, the device comprises a conveyor, a first station for detecting chicks of a first sex, male or female, comprising said camera, and an actuator adapted to pick or eject from the conveyor the chicks detected as belonging to the first sex, wherein the computing unit is configured to implement on the image acquired by the camera a first classification model optimized to detect the first sex, and the computing unit is further configured to implement a second classification model optimized to detect the second sex, on images acquired on chicks not having been determined of the first sex.

The proposed method makes it possible, from one or more images of a chick, to automatically, quickly and reliably determine the sex of the chick. The method comprises in particular a determining of a region of interest of the image on which the wing feathers of the chick are visible, then processing the image making it possible to automatically extract a set of lines corresponding to the feathers, and finally classifying the sex of the chick according to parameters of these lines.

Determining the sex of a chick can thus be carried out in 400 ms, which is much faster than prior methods.

### BRIEF DESCRIPTION OF THE DRAWINGS

Other features, details and advantages will become apparent on reading the detailed description below, and on analyzing the attached drawings, in which:

FIG. 1a already described, shows an example of a male chick wing.

FIG. 1b, already described, shows an example of a female chick wing.

FIG. 2a schematically shows an example of a device for determining the sex of a chick according to one embodiment.

FIG. 2a schematically shows another example of a device for determining the sex of a chick according to one embodiment.

FIG. 3 schematically shows the main steps of a method for determining the sex of a chick according to one embodiment.

FIG. 4a, FIG. 4b show examples of images used for training a classifier used for determining the region of interest of the image.

FIG. 5 shows an example of a Haar feature that can be calculated on the image for determining the region of interest.

FIG. 6a shows a step of an example of processing implemented on a region of interest of an image to extract a set of lines corresponding to the feathers.

FIG. 6b shows a step of an example of processing implemented on a region of interest of an image to extract a set of lines corresponding to the feathers.

FIG. 6c shows a step of an example of processing implemented on a region of interest of an image to extract a set of lines corresponding to the feathers.

FIG. 6d shows a step of an example of processing implemented on a region of interest of an image to extract a set of lines corresponding to the feathers.

FIG. 6e shows a step of an example of processing implemented on a region of interest of an image to extract a set of lines corresponding to the feathers.

FIG. 6f shows a step of an example of processing implemented on a region of interest of an image to extract a set of lines corresponding to the feathers.

FIG. 7 shows a set of lines corresponding to long feathers and short feathers of chicks.

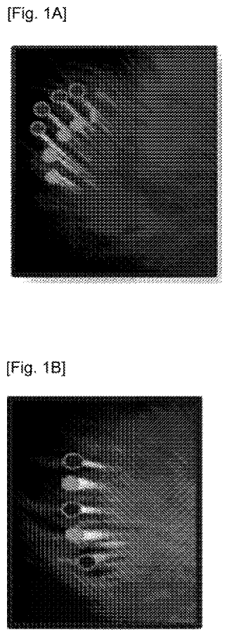

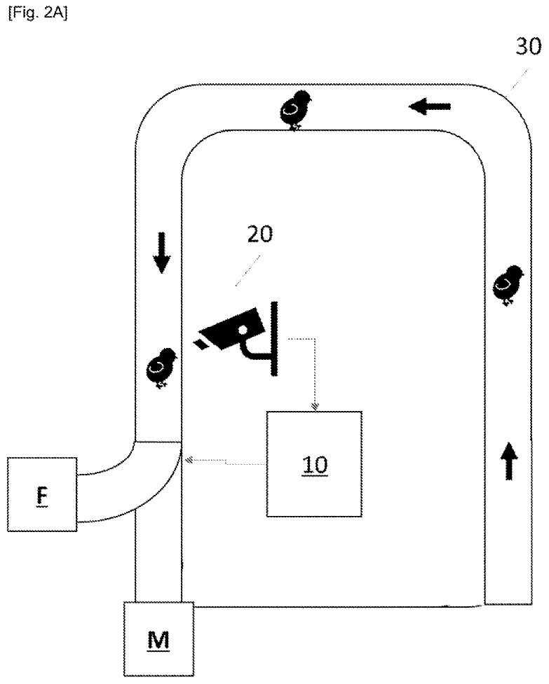

### DESCRIPTION OF EMBODIMENTS

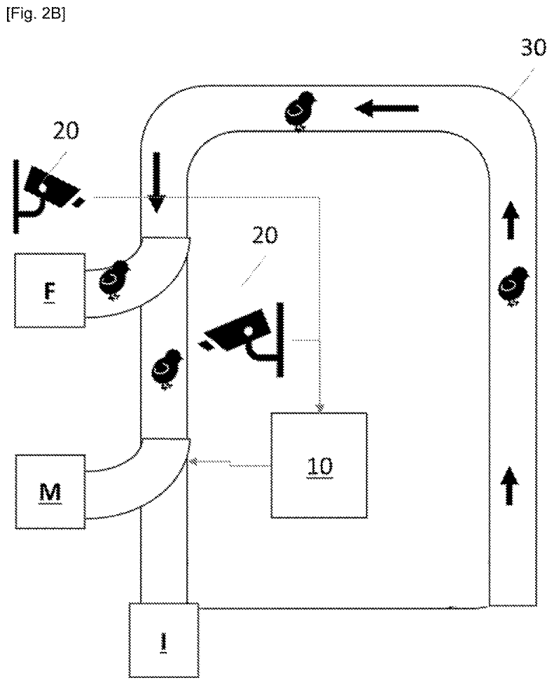

Referring to FIG. 3, a method for determining the sex of a chick will now be described. This method is implemented by a computer 10 which comprises one or more computing unit(s) 11 of the type for example processor, microprocessor, controller, microcontroller, FPGA, etc., and a memory 12 storing code instructions executed by the computing unit. As will be seen in more detail below, the memory 12 can also store pre-trained classification models which are used during the processing.

The method for determining the sex of a chick is implemented by the processing of one or more images representing a wing of the chick received by the computer 10.

In one embodiment shown schematically in FIG. 2a, a device 1 for determining the sex of a chick comprises a camera 20 adapted to acquire images of chicks and a computing unit to which the camera 20 transmits the acquired images.

The camera 20 can be adapted to acquire images in wavelengths between 340 and 500 nm, for example, which is a wavelength range for which the best contrast is obtained to view the feathers of the wings. The camera can be adapted to acquire images in a wavelength range between 400 and 450 nm. The camera can be monochromatic and centered on a wavelength of this range, for example 405 nm.

In some embodiments, the device 1 comprises a conveyor 30 adapted to bring chicks individually and successively into the field of view of the camera 20. The conveyor 30 can be adapted to unbalance the chick so that the chick has its wings unfurled at the moment when it passes in front of the camera. As a non-limiting example, the conveyor 30 may comprise a portion located upstream of the camera 20, this portion being able to vibrate in such a way as to unbalance the chicks.

In addition, the camera can take a series of images of each chick, for example at least 20 consecutively acquired images, for example 40 images of each chick, that the camera 20 transmits to the computer 10 for implementing the method for determining the sex of the chick. The set of images taken by the camera has the same dimensions and the same resolution.

In one embodiment shown in FIG. 2a, the device 1 may comprise a single station making it possible to sort the chicks, which comprises a camera 20 taking the images of the chicks and the computer 10 directly determines whether the chick corresponds to a male or a female, by applying the processing described below, which in particular comprises running a classification algorithm trained to determine whether the chick is a male or a female.

In one variant, as for example the case shown in FIG. 2b, the device 1 can comprise a first station comprising a camera 20 taking the images of the chicks (with their wings unfurled as described above), and transmitting these images to a computer 10 making it possible to determine only whether the chick corresponds to a first sex, male or female. If necessary, the computer 10 implements a classification model optimized to detect the first sex. In the example shown in FIG. 2b, the first station makes it possible to determine only female chicks.

The first station further comprises an adapter adapted to pick up, or eject, from the conveyor the chicks identified as corresponding to the first sex.

For the remaining chicks, in one embodiment, the computer 10 can apply to the corresponding images a second classification model optimized to detect the second sex. In one variant, the device comprises a second station also comprising a camera to retrieve images of the remaining chicks. The computer 10 (which can be distinct or identical from that of the first station) runs the processing described below on the images obtained and applies an optimized model to detect the second sex. The device 1 may comprise a second actuator suitable for collecting or ejector from the conveyor, the chicks identified as corresponding to the second sex.

The remaining chicks can be collected to be delivered to the conveyor starting point or to be analyzed by an operator.

According to yet another variant, the conveyor comprises two image-taking stations, each with at least one camera, making it possible to double the chances of properly presenting the chick with its wings open, and a classification model between the male and female sexes, or two classification models optimized respectively to detect each sex, receive as input the images coming from the two cameras.

Referring back to FIG. 3, the method comprises a step 100 of determining, from an image of the chick, a region of interest of the image on which the feathers of a wing are visible. This step makes it possible on the one hand to discard the images on which the feathers of the wings are not visible, and on the other hand to make it possible to run the remainder of the processing on a region which can be of smaller size than the original image, which therefore makes it possible to reduce the computing time.

From the region of interest of the image thus obtained, the method further comprises, and as described in more detail below, implementing a classification model 400 trained on a training database comprising images of the wings of male chicks and the wings of female chicks, to determine the male or female sex of the chick. As indicated above, the method may comprise successively implementing a first optimized model to determine that a chick belongs to a first sex, male or female, and a second optimized model to determine that a chick belongs to the other sex, female or male.

In some embodiments, determining 100 of the region of interest can be implemented by applying a trained model, for example by a deep learning model.

In a particular embodiment, determining the region of interest of the image comprises scanning the image with a window of determined size, to define a plurality of regions of the image.

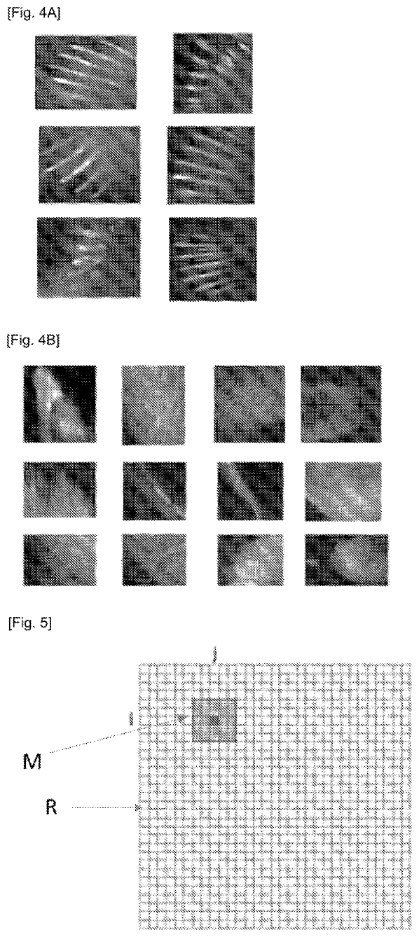

For each region obtained, Haar features are calculated 110, making it possible to obtain a vector associated with each region. Referring to FIG. 5, a Haar feature is obtained from a pattern M of pixels of smaller size than the size of the region R. The pixel pattern comprises two subsets of pixels corresponding to determined locations within the pattern. The Haar feature is calculated by subtracting the cumulative intensities of the pixels of the first subset from the cumulative intensities of the pixels of the second subset. Thus, a Haar feature is obtained for each possible position of the pixel pattern among the region, and the obtained vector gathers all the Haar features calculated over the entire region considered.

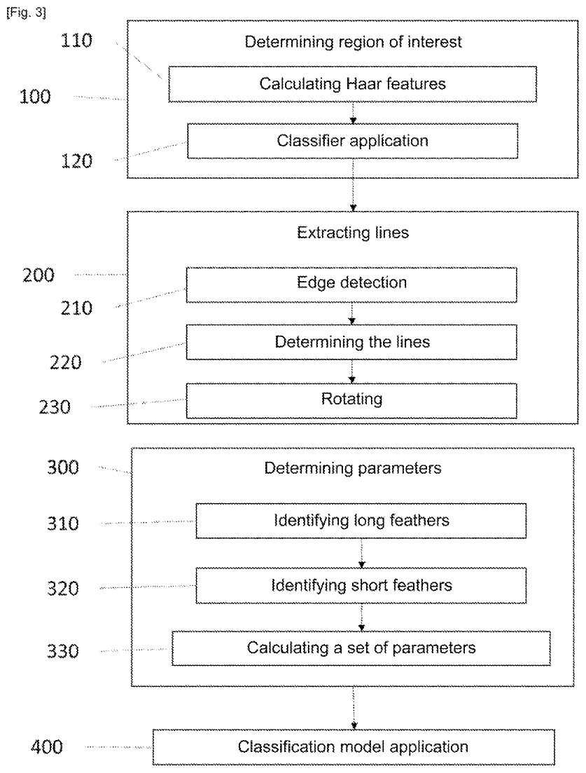

The vector obtained is then supplied to a trained classifier 120 to determine whether the region from which the vector has been obtained is a region representing feathers or not. With reference to FIGS. 4a and 4b, the classifier is advantageously trained by an algorithm of the Adaberost type, on a database of images including images showing feathers (FIG. 4a) and other images (FIG. 4b) showing no feathers, on which Haar feature vectors were calculated according to the preceding paragraph. The trained classifier thus may for example be a decision tree.

The region of interest of the image is the region detected by the classifier as showing feathers.

Multiple regions of interest can be detected in the same image. In some embodiments, the following steps of the method can be implemented for all of the regions of interest detected for an image. Alternatively, the following steps of the method are implemented for a first region of interest, and only in the case where determining the sex of the chick is not possible from this first region of interest, the method is repeated on a second region of interest, and so on until the sex of the chick has been determined from the image.

In some embodiments, once the region(s) of interest have been identified for an image, the method may comprise the direct application 400 of a classification model trained on said region of interest, e.g. of the neural network type.

In one variant, and as shown in FIG. 3, the method comprises, before implementing the classification model, a step 200 of processing the region of interest in order to determine lines corresponding to the feathers of the chick, and a step 300 of determining a set of relevant parameters provided at the input of the model.

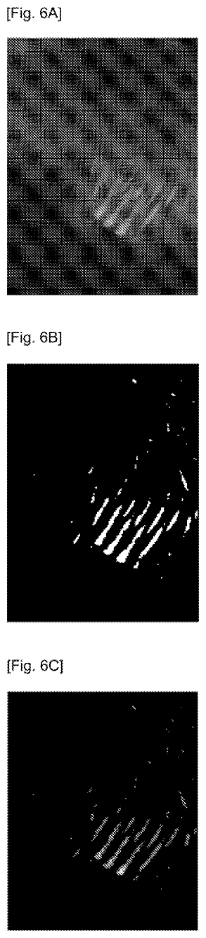

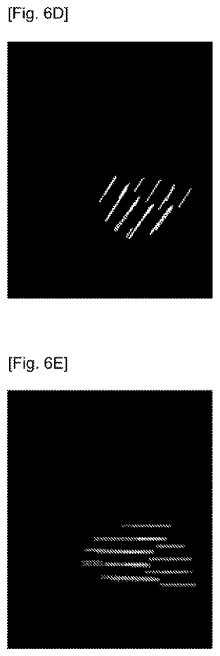

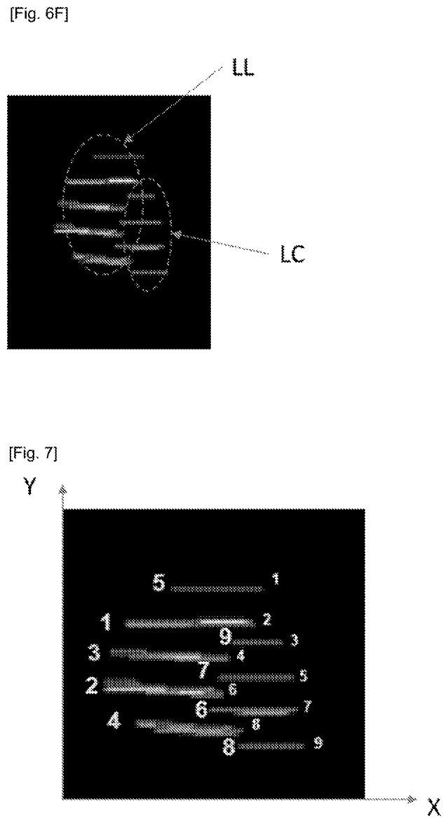

Referring to FIGS. 6a to 6f, we will describe an embodiment of a processing 200 of the (or each) region of interest to extract lines corresponding to the feathers of the chick on the image.

This processing comprises implementing edge detection 210 on the region of interest, and determining a set of lines 220, corresponding to the feathers, from the detected edges.

FIG. 6a shows an example of a region of interest of an image obtained at the end of step 100 described above. In some embodiments, the edge detection processing comprises implementing a Gaussian filter and thresholding the region of interest to obtain a binary representation of said region, as shown in FIG. 6b, that is, a region in which all the pixels are either black or white.

The thresholding is advantageously adaptive so as to compensate for any variations in the lighting conditions of the image. The local threshold can be determined, for each current pixel of the region of interest, from the intensity values of the pixels included in a local vicinity of the current pixel, for example a square window centered on the current pixel. The threshold may for example be the average of the intensities of the pixels of the window.

The edge detection processing can then comprise a step of computing a distance map on the binary representation obtained, to determine a distance between each point of the binary region and an edge closest to the point. The metric used to calculate the distances may for example be the Chebyshev distance or chessboard distance. The distance map obtained is then normalized to obtain a grayscale representation of the region of interest, as in the example shown in FIG. 6c.

The processing then comprises an operation of eroding the obtained region of interest, making it possible to reduce the noise in the image and in particular between the feathers.

The determination 220 of the lines corresponding to the feathers can then be carried out on the region of interest obtained by applying a Hough transform. In the example shown in FIGS. 6a to 6f, the lines obtained are visible in FIG. 6d. It will be understood from the foregoing description that the lines are sets of pixels arranged substantially in line form, but the lines are not necessarily perfectly rectilinear or of constant width.

Once the lines are obtained, the method may also comprise a rotation 230 of the region of interest so that the lines are substantially horizontal. In this respect, the angle of rotation can be determined by identifying the longest feathers of the region of interest, for example the two or three longest feathers, and by calculating the angle of each line relative to the abscissa axis X, and by calculating the average angle over the feathers considered.

In some embodiments, once the rotation has been carried out, the angle of each line corresponding to a feather with respect to the X-axis can be recalculated, and the lines forming an angle, relative to this axis, greater than a determined threshold, are removed because these lines then correspond to noise and not to true feathers. The angular threshold may be between 20 and 30°, for example equal to 25°. An example of a result is shown in FIG. 6e.

Referring back to FIG. 3, the determination of the parameters 300 from the lines representing the feathers first comprises identifying 310 the lines corresponding to long feathers, and identifying the lines 320 corresponding to short feathers. Figure of shows the lines LL correspond to the long feathers, and the lines LC corresponding to short feathers.

The identification of lines corresponding to long feathers 310 is implemented by initializing a set of lines corresponding to long feathers, said set comprising the longest line of all those that appear in the region obtained in step 200.

The set is then completed with other lines that can be determined as follows:
for each line included in the set of lines corresponding to long feathers, identifying all of the neighboring lines of the considered line, along the vertical axis or ordinate axis Y. In this respect, an example is shown in FIG. 7 in which the Y-axis is shown and the lines are indexed from top to bottom, the indexes being shown on the right of each line. The left-hand number of each line represents the ranking of the lines in order of length. Thus, in the example of FIG. 7, the longest line is that of index 2 and its neighbors along the ordinate axis Y are the indexed lines 1, 3 and 4.
Then for each neighboring line, a set of parameters is calculated comprising:
   A length difference between the considered line of the set and the neighboring line,
   A distance between the center of the considered line and the center of the neighboring line, along the abscissa axis X and the ordinate axis Y.
These parameters are compared to predetermined thresholds. The distance threshold is preferably less than 25%, for example between 10 and 25%, for example equal to 15%. The distance threshold between the center of each line along the Y-axis is preferably less than 50% of the length of the considered line (the line corresponding to a long feather), for example equal to 30% of the length of this line. The distance threshold between the center of each line along the X axis is preferably less than 50% of the length of the considered line, preferably less than 30%, for example equal to 25%. If these three parameters are less than their respective thresholds, then the neighboring line is identified as corresponding to a long feather and added to the set.

The method is repeated until no neighboring line is added to the set of lines corresponding to long feathers.

In one embodiment, the other lines are automatically considered to be lines corresponding to short feathers. However, for greater accuracy, the method comprises an identification 320 of the lines corresponding to short feathers. This identification is implemented, for each line corresponding to a long feather of the set formed previously, following the position indexes along the ordinate axis Y, i.e. starting with the line located at the highest vertical position of the set, and comprises:
identifying, among the lines not belonging to the set of lines corresponding to long feathers, the neighboring lines of the considered line. In the example of FIG. 7, no neighbor would be identified for the line with index 1, but the neighboring line with index 3 for the line with index 2 would be identified.
For each line adjacent to the line in question, a length difference between the considered line and the neighboring line is then calculated, a distance along the vertical axis between the two lines, and a distance along the horizontal axis between a proximal or distal end of the considered line and a distal end, respectively proximal end of the neighboring line, according to the relative position on the axis X of the longest lines relative to the shortest lines.

The term “distal end” refers to the end furthest from the origin of the marker along the X-axis, and by “proximal end”, the closest end. If, as in the example of FIG. 7, the shortest lines of the region obtained after the processing 200 are located in the vicinity of the distal ends of the longest lines, then a distance is calculated between the distal end of the line in question and the proximal end of the neighboring line. If, conversely, the shortest lines are close to the proximal ends of the longest lines, a distance is calculated between the proximal end of the line in question and the distal end of the neighboring line.
These parameters are compared to predetermined thresholds. The threshold for the difference in length is preferably between 50 and 80% of the length of the considered line, for example equal to 60%. The distance threshold along the ordinate axis Y is preferably less than 50% of the length of the considered line, for example equal to 30%. The only distance along the X-axis between the proximal, respectively distal end of the considered line and the distal, respectively proximal, end of the neighboring line is less than 50% of the length of the considered line, for example equal to 50%. If these three parameters are less than their respective thresholds, then the neighboring line is identified as corresponding to a short feather.

Once the lines correspond to long feathers and the lines corresponding to short feathers are identified, the method may comprise a calculation 330 of a set of parameters from the region of interest from the processing 200, these parameters then being provided to a classification model trained to determine the male or female sex of the chick.

In some embodiments, these parameters comprise at least the number of rows corresponding to long feathers and the number of rows corresponding to short feathers.

In addition, the parameters may further comprise an average angle between the lines and the horizontal, and a deviation or an average distance, measured vertically (axis Y) between two adjacent lines.

In some embodiments, the parameters used for the model may further comprise one or more or any combination of all of the following parameters:
the minimum, maximum, and/or average horizontal and/or vertical positions of the centers of the lines,
the minimum, average and/or maximum horizontal and/or vertical distance between the centers of two consecutive lines,
the minimum, average and/or maximum lengths of the lines corresponding to long feathers,
the minimum, average and/or maximum lengths of the lines corresponding to short feathers,
the average intensity of the pixels of a line corresponding to a short feather,
the average intensity of the pixels of a line corresponding to a long feather,
the difference in average intensity of the pixels between the lines corresponding to long feathers and the lines corresponding to short feathers,
the location relative to the horizontal axis of each line,
a minimum, average and/or maximum angle between two lines,
a minimum, average and/or maximum distance between the proximal ends of two lines corresponding to a long feather and one line corresponding to a short feather located between them,
a minimum, average and/or maximum distance between the distal ends of two lines corresponding to a long feather and one line corresponding to a short feather located between them.

The parameters calculated at the end of step 300 are provided to a model trained to determine the sex of the chick, the model being trained so as to have two output classes, male/female, or alternatively, two classes comprising a first sex and an indeterminate class.

The model used is for example, but not limited to a decision tree.

As indicated above, the model is trained on a database of annotated images of chick wings. Preferably, the annotated images are images that have undergone the line extraction processing of step 200, and for which the steps of determining the lines corresponding to long feathers and lines corresponding to short feathers, and of extracting the parameters 300 are also implemented, so that these parameters can be provided to the model for its training.

The annotation, that is to say the attribution to the region of interest considered of a male or female nature of the chick, is carried out by an experienced operator as a function of the number of long feathers and short feathers of the considered region of interest.

This annotation can also comprise a degree of certainty associated with the determined sex, which can also be indicated by the operator. For example, on an image comprising 5 long feathers and 0 short feathers, the annotation can be “male; 100%”. According to another example, on an image comprising 4 long feathers and 2 short feathers, the annotation can be “female; 100%”. The degree of certainty is preferably between 60 and 100% and in the more uncertain cases, the annotation does not determine sex. According to a third example, on an image comprising 3 long feathers and 1 short feather, the annotation can be “indeterminate”. This type of image is then not preserved for training the model.

In one embodiment in which several images are acquired for the same chick, for example at least 20 images, the processing described above can be implemented for each image. Thus, for each image, a result is obtained concerning the sex of the chick, and the sex of the chick is then determined by a majority vote, i.e. the most frequently obtained result, among the results obtained for all the images of the same chick.

In one variant, the parameters extracted from each image can be provided to the trained model, and determining the sex of the chick is carried out by a majority vote,

Experimental results concerning the application of the method described above including the implementation of steps 200 and 300 and applying a trained model of the decision tree type, on an equal-sex population of 10,000 chicks, are reproduced below. The parameters extracted from the images and used in this experiment are:
the number of long and short feathers,
the average angle between the lines and the horizontal, and
the average distance along the Y-axis between two adjacent lines, and
the set of additional parameters listed above in paragraph 92.

TABLE 1

| Females | Females detected | Unknown | Error rate | Detection rate |
|---|---|---|---|---|
| 5000 | 4350 | 650 | 4.5% | 86.99% |
| Males | Males detected | Unknown | Error rate | Detection rate |
| 5000 | 3500 | 1500 | 4.2% | 70% |
| Total | Correct detections | Unknown | Error rate | Detection rate |
| 10000 | 7850 | 2150 | 4.35% | 78.5% |

1. A method for determining the sex of a chick, the method being implemented by computer from an image of a chick, the method comprising:
determining (100) a region of interest of the image on which the feathers of a wing are visible,
running, on said region of interest, a classification model (400) trained on a training data set comprising images of male chick wings and of female chick wings, to determine the male or female sex of the chick.

2. The method according to claim 1, the method being implemented for each of a plurality of images acquired on a same chick, and further comprising a step of determining the sex of the chick from the results obtained by the classification model for all of the images.

3. The method according to claim 1, wherein the determining a region of interest of the image (100) comprises:
scanning the image with a window of determined size to define a plurality of regions of the image,
for each region, calculating a Haar feature of the region (110),
the application on each Haar feature of a trained classifier (120) to determine whether or not the region represents feathers, and
determining a region of interest of the image as a region representing feathers.

4. The method according to claim 1, further comprising processing the region of interest (200) to determine a set of lines corresponding to the feathers of the chick on the image, determining (300) of a set of parameters from the extracted lines, and the classification model is applied (400) to said set of parameters.

5. The method according to claim 4, wherein the processing of the region of interest (200) to determine a set of lines corresponding to the feathers on the image, comprises:
running an edge detection processing (210) on the region of interest, and
applying, to the edges resulting from the processing, a Hough transform to determine a set of lines (220) corresponding to the feathers visible on the region of interest.

6. The method of claim 4, wherein determining the parameters (300) from the extracted lines comprises:
identifying a set of lines corresponding to long feathers (310), and
identifying a set of lines corresponding to short feathers (320).

7. The method according to claim 6, comprising rotating (230) the region of interest so that the lines representing the feathers extend substantially horizontally, ranking each line in order of length, and identifying the set of lines corresponding to long feathers (310) comprises:
initializing the set of lines corresponding to long feathers, said set comprising the longest line,
implementing, for each line included in said set, the following steps:
identifying all the neighboring lines of the considered line along the vertical axis,
calculating, for each neighboring line, a length difference and a distance between the center of the neighboring line and the center of the line in question,
if the relative difference and the distance are less than respective thresholds, identifying the neighboring line as a line corresponding to a long feather, and adding to the set of lines corresponding to long feathers.

8. A method for identifying a set of lines corresponding to short feathers (320) comprises implementing, for each line corresponding to a long feather of the set, starting with the line located at the maximum vertical position of the set, the following steps:
identifying, among the lines not belonging to the set of lines corresponding to long feathers, the neighboring lines of the considered line,
calculating, for each neighboring line, a length difference, a distance along the vertical axis between the considered line and the neighboring line, and a distance along the horizontal axis between a distal end, respectively proximal end, of the considered line, and the proximal end, respectively distal end, of the neighboring line.
if the calculated differences and distances are less than respective thresholds, identifying the neighboring line as a line corresponding to a short feather.

9. The method according to claim 4, wherein the parameters determined from the lines comprise at least:
a number of lines corresponding to long feathers,
a number of lines corresponding to short feathers,
an average angle between the lines and the horizontal, and
an average deviation, measured vertically, between two adjacent lines.

10. The method according to claim 4, wherein the classification model is trained on a database of annotated training images, where each training image is obtained by applying steps of determining a region of interest and processing the region of interest to determine a set of lines representing the feathers, and of extracting parameters from said lines, and the annotation comprises an indication of the sex of the chick and an associated certainty level, determined from a number of lines corresponding to long feathers and a number of lines corresponding to short feathers.

11. The method according to claim 1, the method being implemented on a set of images of the same chick, and comprises determining the sex of the chick from the result most frequently provided by the classification model.

12. A computer program product, comprising code instructions for implementing the method according to claim 1, when executed by a computing unit.

13. A device (1) for determining the sex of a chick comprising at least:
a camera (20) adapted to acquire at least one image of a chick, and
a computing unit (10) configured to implement the method according to claim 1 on the image acquired by the camera.

14. The device (1) according to claim 13, further comprising a conveyor (30) adapted to bring a chick into the field of view of the camera (20), wherein the conveyor is adapted to unbalance the chicks so that the chick has its wings unfurled when it is in front of the camera.

15. The device (1) according to claim 13, comprising conveyor (30), a first station for detecting chicks of a first sex, male or female, comprising said camera (20), and an actuator adapted to pick or eject from the conveyor the chicks detected as belonging to the first sex, wherein the computing unit (10) is configured to implement on the image acquired by the camera a first classification model optimized to detect the first sex, and the computing unit (10) is further configured to implement a second classification model optimized to detect the second sex, on images acquired on chicks not having been determined of the first sex.
